SEQUENCE LISTING (7 sequences)

SEQ ID NO: 1 - protein, 1445 aa, Homo sapiens
ATRRYYLGAVELSWDYMQSDLGELPVDARFPPRVPKSFPFNTSVVYKKTLFVEFTDHLFNIAKPRPPWMGLLGPTIQAEVYDTVVITLKNMASHPVSLHAVGVSYWKASEGAEYDDQTSQREKEDDKVFPGGSHTYVWQVLKENGPMASDPLCLTYSYLSHVDLVKDLNSGLIGALLVCREGSLAKEKTQTLHKFILLFAVFDEGKSWHSETKNSLMQDRDAASARAWPKMHTVNGYVNRSLPGLIGCHRKSVYWHVIGMGTTPEVHSIFLEGHTFLVRNHRQASLEISPITFLTAQTLLMDLGQFLLFCHISSHQHDGMEAYVKVDSCPEEPQLRMKNNEEAEDYDDDLTDSEMDVVRFDDDNSPSFIQIRSVAKKHPKTWVHYIAAEEEDWDYAPLVLAPDDRSYKSQYLNNGPQRIGRKYKKVRFMAYTDETFKTREAIQHESGILGPLLYGEVGDTLLIIFKNQASRPYNIYPHGITDVRPLYSRRLPKGVKHLKDFPILPGEIFKYKWTVTVEDGPTKSDPRCLTRYYSSFVNMERDLASGLIGPLLICYKESVDQRGNQIMSDKRNVILFSVFDENRSWYLTENIQRFLPNPAGVQLEDPEFQASNIMHSINGYVFDSLQLSVCLHEVAYWYILSIGAQTDFLSVFFSGYTFKHKMVYEDTLTLFPFSGETVFMSMENPGLWILGCHNSDFRNRGMTALLKVSSCDKNTGDYYEDSYEDISAYLLSKNNAIEPRSFSQNSRHPSQNPPVLKRHQREITRTTLQSDQEEIDYDDTISVEMKKEDFDIYDEDENQSPRSFQKKTRHYFIAAVERLWDYGMSSSPHVLRNRAQSGSVPQFKKVVFQEFTDGSFTQPLYRGELNEHLGLLGPYIRAEVEDNIMVTFRNQASRPYSFYSSLISYEEDQRQGAEPRKNFVKPNETKTYFWKVQHHMAPTKDEFDCKAWAYFSDVDLEKDVHSGLIGPLLVCHTNTLNPAHGRQVTVQEFALFFTIFDETKSWYFTENMERNCRAPCNIQMEDPTFKENYRFHAINGYIMDTLPGLVMAQDQRIRWYLLSMGSNENIHSIHFSGHVFTVRKKEEYKMALYNLYPGVFETVEMLPSKAGIWRVECLIGEHLHAGMSTLFLVYSNKCQTPLGMASGHIRDFQITASGQYGQWAPKLARLHYSGSINAWSTKEPFSWIKVDLLAPMIIHGIKTQGARQKFSSLYISQFIIMYSLDGKKWQTYRGNSTGTLMVFFGNVDSSGIKHNIFNPPIIARYIRLHPTHYSIRSTLRMELMGCDLNSCSMPLGMESKAISDAQITASSYFTNMFATWSPSKARLHLQGRSNAWRPQVNNPKEWLQVDFQKTMKVTGVTTQGVKSLLTSMYVKEFLISSSQDGHQWTLFFQNGKVKVFQGNQDSFTPVVNSLDPPLLTRYLRIHPQSWVHQIALRMEVLGCEAQDLY

SEQ ID NO: 2 - protein, 320 aa, homo sapiens
MAQVLRGTVTDFPGFDERADAETLRKAMKGLGTDEESILTLLTSRSNAQRQEISAAFKTLFGRDLLDDLKSELTGKFEKLIVALMKPSRLYDAYELKHALKGAGTNEKVLTEIIASRTPEELRAIKQVYEEEYGSSLEDDVVGDTSGYYQRMLVVLLQANRDPDAGIDEAQVEQDAQALFQAGELKWGTDEEKFITIFGTRSVSHLRKVFDKYMTISGFQIEETIDRETSGNLEQLLLAVVKSIRSIPAYLAETLYYAMKGAGTDDHTLIRVMVSRSEIDLFNIRKEFRKNFATSLYSMIKGDTSGDYKKALLLLCGEDD

SEQ ID NO: 3 - protein, 364 aa, homo sapiens
LDICSKNPCHNGGLCEEISQEVRGDVFPSYTCTCLKGYAGNHCETKCVEPLGMENGNIANSQIAASSVRVTFLGLQHWVPELARLNRAGMVNAWTPSSNDDNPWIQVNLLRRMWVTGVVTQGASRLASHEYLKAFKVAYSLNGHEFDFIHDVNKKHKEFVGNWNKNAVHVNLFETPVEAQYVRLYPTSCHTACTLRFELLGCELNGCANPLGLKNNSIPDKQITASSSYKTWGLHLFSWNPSYARLDKQGNFNAWVAGSYGNDQWLQVDLGSSKEVTGIITQGARNFGSVQFVASYKVAYSNDSANWTEYQDPRTGSSKIFPGNWDNHSHKKNLFETPILARYVRILPVAWHNRIALRLELLGC

SEQ ID NO: 4 - protein, 684 aa, homo sapiens
EITRTTLQSDQEEIDYDDTISVEMKKEDFDIYDEDENQSPRSFQKKTRHYFIAAVERLWDYGMSSSPHVLRNRAQSGSVPQFKKVVFQEFTDGSFTQPLYRGELNEHLGLLGPYIRAEVEDNIMVTFRNQASRPYSFYSSLISYEEDQRQGAEPRKNFVKPNETKTYFWKVQHHMAPTKDEFDCKAWAYFSDVDLEKDVHSGLIGPLLVCHTNTLNPAHGRQVTVQEFALFFTIFDETKSWYFTENMERNCRAPCNIQMEDPTFKENYRFHAINGYIMDTLPGLVMAQDQRIRWYLLSMGSNENIHSIHFSGHVFTVRKKEEYKMALYNLYPGVFETVEMLPSKAGIWRVECLIGEHLHAGMSTLFLVYSNKCQTPLGMASGHIRDFQITASGQYGQWAPKLARLHYSGSINAWSTKEPFSWIKVDLLAPMIIHGIKTQGARQKFSSLYISQFIIMYSLDGKKWQTYRGNSTGTLMVFFGNVDSSGIKHNIFNPPIIARYIRLHPTHYSIRSTLRMELMGCDLNSCSMPLGMESKAISDAQITASSYFTNMFATWSPSKARLHLQGRSNAWRPQVNNPKEWLQVDFQKTMKVTGVTTQGVKSLLTSMYVKEFLISSSQDGHQWTLFFQNGKVKVFQGNQDSFTPVVNSLDPPLLTRYLRIHPQSWVHQIALRMEVLGCEAQDLY

SEQ ID NO: 5 - protein, 158 aa, homo sapiens
CANPLGLKNNSIPDKQITASSSYKTWGLHLFSWNPSYARLDKQGNFNAWVAGSYGNDQWLQVDLGSSKEVTGIITQGARNFGSVQFVASYKVAYSNDSANWTEYQDPRTGSSKIFPGNWDNHSHKKNLFETPILARYVRILPVAWHNRIALRLELLGC

SEQ ID NO: 6 - protein, 153 aa, homo sapiens
CSMPLGMESKAISDAQITASSYFTNMFATWSPSKARLHLQGRSNAWRPQVNNPKEWLQVDFQKTMKVTGVTTQGVKSLLTSMYVKEFLISSSQDGHQWTLFFQNGKVKVFQGNQDSFTPVVNSLDPPLLTRYLRIHPQSWVHQIALRMEVLGC

SEQ ID NO: 7 - protein, 392 aa, artificial
  FLAG-lactadherin
MPLLLLLPLLWAGALADYKDDDDKGGGSLDICSKNPCHNGGLCEEISQEVRGDVFPSYTCTCLKGYAGNHCETKCVEPLGMENGNIANSQIAASSVRVTFLGLQHWVPELARLNRAGMVNAWTPSSNDDNPWIQVNLLRRMWVTGVVTQGASRLASHEYLKAFKVAYSLNGHEFDFIHDVNKKHKEFVGNWNKNAVHVNLFETPVEAQYVRLYPTSCHTACTLRFELLGCELNGCANPLGLKNNSIPDKQITASSSYKTWGLHLFSWNPSYARLDKQGNFNAWVAGSYGNDQWLQVDLGSSKEVTGIITQGARNFGSVQFVASYKVAYSNDSANWTEYQDPRTGSSKIFPGNWDNHSHKKNLFETPILARYVRILPVAWHNRIALRLELLGC

(19) United States
(12) Patent Application Publication
(10) Pub. No.: US 2014/0308707 A1
(43) Pub. Date: Oct. 16, 2014

(54) METHOD FOR PRODUCTION OF FACTOR VIII

(71) Applicant: Novo Nordisk A/S, Bagsvaerd (DK)

(72) Inventors: Laust Bruun Johnsen, Bagsvaerd (DK); Mille Petersen Kolind, Maaloev (DK); Peder Lisby Noerby, Bagsvaerd (DK)

(21) Appl. No.: 14/358,886

(22) PCT Filed: Nov. 2, 2012
(86) PCT No.: PCT/EP2012/071701

§ 371 Date: May 16, 2014

Related U.S. Application Data
(60) Provisional application No. 61/563,188, filed on Nov. 23, 2011.

(30) Foreign Application Priority Data
Nov. 21, 2011 (EP) 11189861.5

(51) Int. Cl. C07K 14/755

(52) U.S. Cl. CPC C07K 14/755
USPC 435/70.3; 435/404

(57) ABSTRACT

The present invention relates to methods of producing a Factor VIII polypeptide in mammalian cell cultures.

### FIELD OF THE INVENTION

The present invention relates to methods for the production of a Factor VIII polypeptide.

### BACKGROUND TO THE INVENTION

Factor VIII is an essential blood clotting factor. Mutations in the Factor VIII gene that result in decreased or defective Factor VIII protein give rise to the genetic disease, haemophilia A, which is characterised by recurrent bleeding episodes. Treatment of haemophilia A requires intravenous infusion of either plasma-derived or recombinant Factor VIII.

Although plasma derived Factor VIII can be used to treat haemophilia, there have been a number of problems with this approach, including the transmission of viruses to patients. Therefore, it is preferable to administer Factor VIII that has been recombinantly expressed.

Large amounts of Factor VIII are difficult to obtain from cell culture. Factor VIII is known to be expressed at very low levels in mammalian cells. Also, Factor VIII is known to be an unstable protein in serum-free or protein-free medium. Addition of various substances has been used to improve the yields of recombinantly produced Factor VIII. For example, using buffers of high strength increases the yield of Factor VIII. However, this harsh treatment does not allow for subsequent re-use of the cells.

Despite insights into Factor VIII regulation, yields of Factor VIII continue to be significantly lower than other recombinant proteins in the heterologous systems used in commercial manufacture. WO 2008/135501 discloses obtaining improved yields of Factor VIII using a ligand that binds to the C2 domain of Factor VIII (for example, Ortho-Phospho-L-serine (OPLS)). However, methods and compositions are needed to further increase yields of Factor VIII which can be isolated from cell culture.

### SUMMARY OF THE INVENTION

Surprisingly, the present inventors have found that by contacting culture cells with an agent that binds to phosphatidylserine, the amount of Factor VIII released into the culture medium and subsequently harvested is substantially increased. In particular, the yield of Factor VIII is significantly increased compared to the yield seen when OPLS, an agent that binds the C2 domain of Factor VIII, is added to the culture medium.

Accordingly, the present invention provides a method for the production of a Factor VIII polypeptide, which method comprises:
a) culturing a mammalian cell capable of expressing a Factor VIII polypeptide under conditions such that the said polypeptide is expressed; and
b) during or after step (a), contacting the said cell with an agent that binds to phosphatidylserine.

The invention further provides:
a cell culture medium that is serum free and comprises i) an agent selected from lactadherin, annexin V, an antiphospholipid antibody and Factor VIII light chain, and ii) Ortho-Phospho-L-serine (OPLS) or an anti-apoptotic protein.
use of a compound capable of binding to phosphatidylserine for increasing the yield of Factor VIII that can be isolated from a mammalian cell culture.

### Sequences

(SEQ ID NO: 1 human B-domain deleted Factor VIII):
ATRRYYLGAVELSWDYMQSDLGELPVDARFPPRVPKSFPFNTSVVYKKTL
FVEFTDHLFNIAKPRPPWMGLLGPTIQAEVYDTVVITLKNMASHPVSLHA
VGVSYWKASEGAEYDDQTSQREKEDDKVFPGGSHTYVWQVLKENGPMASD
PLCLTYSYLSHVDLVKDLNSGLIGALLVCREGSLAKEKTQTLHKFILLFA
VFDEGKSWHSETKNSLMQDRDAASARAWPKMHTVNGYVNRSLPGLIGCHR
KSVYWHVIGMGTTPEVHSIFLEGHTFLVRNHRQASLEISPITFLTAQTLL
MDLGQFLLFCHISSHQHDGMEAYVKVDSCPEEPQLRMKNNEEAEDYDDDL
TDSEMDVVRFDDDNSPSFIQIRSVAKKHPKTWVHYIAAEEEDWDYAPLVL
APDDRSYKSQYLNNGPQRIGRKYKKVRFMAYTDETFKTREAIQHESGILG
PLLYGEVGDTLLIIFKNQASRPYNIYPHGITDVRPLYSRRLPKGVKHLKD
FPILPGEIFKYKWTVTVEDGPTKSDPRCLTRYYSSFVNMERDLASGLIGP
LLICYKESVDQRGNQIMSDKRNVILFSVFDENRSWYLTENIQRFLPNPAG
VQLEDPEFQASNIMHSINGYVFDSLQLSVCLHEVAYWYILSIGAQTDFLS
VFFSGYTFKHKMVYEDTLTLFPFSGETVFMSMENPGLWILGCHNSDFRNR
GMTALLKVSSCDKNTGDYYEDSYEDISAYLLSKNNAIEPRSFSQNSRHPS
QNPPVLKRHQREITRTTLQSDQEEIDYDDTISVEMKKEDFDIYDEDENQS
PRSFQKKTRHYFIAAVERLWDYGMSSSPHVLRNRAQSGSVPQFKKVVFQE
FTDGSFTQPLYRGELNEHLGLLGPYIRAEVEDNIMVTFRNQASRPYSFYS
SLISYEEDQRQGAEPRKNFVKPNETKTYFWKVQHHMAPTKDEFDCKAWAY
FSDVDLEKDVHSGLIGPLLVCHTNTLNPAHGRQVTVQEFALFFTIFDETK
SWYFTENMERNCRAPCNIQMEDPTFKENYRFHAINGYIMDTLPGLVMAQD
QRIRWYLLSMGSNENIHSIHFSGHVFTVRKKEEYKMALYNLYPGVFETVE
MLPSKAGIWRVECLIGEHLHAGMSTLFLVYSNKCQTPLGMASGHIRDFQI
TASGQYGQWAPKLARLHYSGSINAWSTKEPFSWIKVDLLAPMIIHGIKTQ
GARQKFSSLYISQFIIMYSLDGKKWQTYRGNSTGTLMVFFGNVDSSGIKH
NIFNPPIIARYIRLHPTHYSIRSTLRMELMGCDLNSCSMPLGMESKAISD
AQITASSYFTNMFATWSPSKARLHLQGRSNAWRPQVNNPKEWLQVDFQKT
MKVTGVTTQGVKSLLTSMYVKEFLISSSQDGHQWTLFFQNGKVKVFQGNQ
DSFTPVVNSLDPPLLTRYLRIHPQSVVVHQIALRMEVLGCEAQDLY
SEQ ID NO: 2 (human annexin V):
MAQVLRGTVTDFPGFDERADAETLRKAMKGLGTDEESILTLLTSRSNAQR
QEISAAFKTLFGRDLLDDLKSELTGKFEKLIVALMKPSRLYDAYELKHAL
KGAGTNEKVLTEIIASRTPEELRAIKQVYEEEYGSSLEDDVVGDTSGYYQ
RMLVVLLQANRDPDAGIDEAQVEQDAQALFQAGELKWGTDEEKFITIFGT
RSVSHLRKVFDKYMTISGFQIEETIDRETSGNLEQLLLAVVKSIRSIPAY
LAETLYYAMKGAGTDDHTLIRVMVSRSEIDLFNIRKEFRKNFATSLYSMI
KGDTSGDYKKALLLLCGEDD
SEQ ID NO: 3 (human lactadherin):
LDICSKNPCHNGGLCEEISQEVRGDVFPSYTCTCLKGYAGNHCETKCVEP
LGMENGNIANSQIAASSVRVTFLGLQHWVPELARLNRAGMVNAWTPSSND
DNPWIQVNLLRRMWVTGVVTQGASRLASHEYLKAFKVAYSLNGHEFDFIH
DVNKKHKEFVGNWNKNAVHVNLFETPVEAQYVRLYPTSCHTACTLRFELL
GCELNGCANPLGLKNNSIPDKQITASSSYKTWGLHLFSWNPSYARLDKQG
NFNAWVAGSYGNDQWLQVDLGSSKEVTGIITQGARNFGSVQFVASYKVAY
SNDSANWTEYQDPRTGSSKIFPGNWDNHSHKKNLFETPILARYVRILPVA
WHNRIALRLELLGC
SEQ ID NO: 4 (human Factor VIII light chain):
EITRTTLQSDQEEIDYDDTISVEMKKEDFDIYDEDENQSPRSFQKKTRHY
FIAAVERLWDYGMSSSPHVLRNRAQSGSVPQFKKVVFQEFTDGSFTQPLY
RGELNEHLGLLGPYIRAEVEDNIMVTFRNQASRPYSFYSSLISYEEDQRQ
GAEPRKNFVKPNETKTYFWKVQHHMAPTKDEFDCKAWAYFSDVDLEKDVH
SGLIGPLLVCHTNTLNPAHGRQVTVQEFALFFTIFDETKSWYFTENMERN
CRAPCNIQMEDPTFKENYRFHAINGYIMDTLPGLVMAQDQRIRWYLLSMG
SNENIHSIHFSGHVFTVRKKEEYKMALYNLYPGVFETVEMLPSKAGIWRV
ECLIGEHLHAGMSTLFLVYSNKCQTPLGMASGHIRDFQITASGQYGQWAP
KLARLHYSGSINAWSTKEPFSWIKVDLLAPMIIHGIKTQGARQKFSSLYI
SQFIIMYSLDGKKWQTYRGNSTGTLMVFFGNVDSSGIKHNIFNPPIIARY
IRLHPTHYSIRSTLRMELMGCDLNSCSMPLGMESKAISDAQITASSYFTN
MFATWSPSKARLHLQGRSNAWRPQVNNPKEWLQVDFQKTMKVTGVTTQGV
KSLLTSMYVKEFLISSSQDGHQWTLFFQNGKVKVFQGNQDSFTPVVNSLD
PPLLTRYLRIHPQSWVHQIALRMEVLGCEAQDLY
SEQ ID NO: 5 (residues 207-364 of
the C2 domain of human lactadherin):
CANPLGLKNNSIPDKQITASSSYKTWGLHLFSWNPSYARLDKQGNFNAWV
AGSYGNDQWLQVDLGSSKEVTGIITQGARNFGSVQFVASYKVAYSNDSAN
WTEYQDPRTGSSKIFPGNWDNHSHKKNLFETPILARYVRILPVAWHNRIA
LRLELLGC
SEQ ID NO: 6 (C2 domain of human Factor
VIII light chain):
CSMPLGMESKAISDAQITASSYFTNMFATWSPSKARLHLQGRSNAWRPQV
NNPKEWLQVDFQKTMKVTGVTTQGVKSLLTSMYVKEFLISSSQDGHQWTL
FFQNGKVKVFQGNQDSFTPVVNSLDPPLLTRYLRIHPQSWVHQIALRMEV
LGC
SEQ ID NO 7: CD33-FLAG-Lactadherin
MPLLLLLPLLWAGALADYKDDDDKGGGSLDICSKNPCHNGGLCEEISQEV
RGDVFPSYTCTCLKGYAGNHCETKCVEPLGMENGNIANSQIAASSVRVTF
LGLQHWVPELARLNRAGMVNAWTPSSNDDNPWIQVNLLRRMWVTGVVTQG
ASRLASHEYLKAFKVAYSLNGHEFDFIHDVNKKHKEFVGNWNKNAVHVNL
FETPVEAQYVRLYPTSCHTACTLRFELLGCELNGCANPLGLKNNSIPDKQ
ITASSSYKTWGLHLFSWNPSYARLDKQGNFNAWVAGSYGNDQWLQVDLGS
SKEVTGIITQGARNFGSVQFVASYKVAYSNDSANWTEYQDPRTGSSKIFP
GNWDNHSHKKNLFETPILARYVRILPVAWHNRIALRLELLGC

### DETAILED DESCRIPTION OF THE INVENTION

The present invention derives from the unexpected finding that contacting mammalian cells expressing a Factor VIII polypeptide with an agent that bind to phosphatidylserine substantially increases the yield of Factor VIII that can be harvested from the culture cell medium. The present invention thus relates to methods for the production of a Factor VIII polypeptide, comprising a) culturing a mammalian cell capable of expressing a Factor VIII polypeptide under conditions such that the said polypeptide is expressed; and b) during or after step (a), contacting the said cell with an agent that binds to phosphatidylserine.

### A Factor VIII Polypeptide

The mature human Factor VIII molecule consists of 2332 amino acids which can be grouped into three homologous A domains, two homologous C domains and a B domain which are arranged in the order: A1-A2-B-A3-C1-C2. A Factor VIII molecule consisting of the heavy chain (HC) and light chain (LC) of Factor VIII connected with a small linker derived from the B-domain (B-domain deleted Factor VIII or BDD-FVIII) retains the biological activity of full length (native) Factor VIII.

As used herein, “Factor VIII polypeptide” encompasses, without limitation, Factor VIII, as well as Factor VIII-related polypeptides, preferably human Factor VIII.

“Factor VIII polypeptide” includes polypeptides having the amino acid sequence as described in Toole et al., Nature 1984, 312: 342-347 (wild-type human Factor VIII), as well as wild-type Factor VIII derived from other species, such as, e.g., bovine, porcine, canine, murine, and salmon Factor VIII. Preferably, the Factor VIII polypeptide is a human Factor VIII polypeptide. Most preferably, the human Factor VIII polypeptide is B-domain deleted/truncated human Factor VIII.

Factor VIII-related polypeptides, including variants, encompass those that exhibit at least about 10%, at least about 20%, at least about 30%, at least about 40%, at least about 50%, at least about 60%, at least about 70%, at least about 80%, at least about 90%, at least about 100%, at least about 110%, at least about 120%, and at least about 130%, of the specific activity of wild-type factor VIII that has been produced in the same cell type, when tested in an assay for the biological activity of Factor VIII.

Tests for the biological activity of Factor VIII are well known in the art. For example, one technique involves testing the ability of a sample of Factor VIII to stimulate the activation of Factor X by Factor IXa in the presence of calcium and phospholipids.

The polypeptide sequence of a B-domain deleted human Factor VIII is given in SEQ ID NO:1.

### Vectors

The nucleic acid molecules encoding Factor VIII may be provided in the form of an expression cassette which includes control sequences operably linked to the inserted sequence, thus allowing for expression of the polypeptide of the invention in vivo in a targeted cell. These expression cassettes, in turn, are typically provided within vectors (e.g., plasmids or recombinant viral vectors). Thus, a polypeptide for use in the invention may be obtained by delivering such a vector to a cell and allowing transcription from the vector to occur.

### Mammalian Host Cell

The methods of the present invention involve production of Factor VIII in a mammalian cell. Any mammalian host cell suitable for production of Factor VIII in culture may be used. For example, the host cell may derived from a human, murine or rodent cell. The host cell may also be used to express polypeptides of interest other than Factor VIII. For instance, a polypeptide capable of binding phosphatidylserine may be contacted with a mammalian cell expressing Factor VIII by co-expressing it with Factor VIII.

Where a cell line is used in which more than one polypeptide of interest, for example both a Factor VIII polypeptide and a polypeptide capable of binding phosphatidylserine, are heterologously expressed, these proteins may be expressed from a single vector or from two separate vectors. More than one copy of the protein encoding sequences may be present in the vector.

Currently preferred cells are HEK293, COS, Chinese Hamster Ovary (CHO) cells, Baby Hamster Kidney (BHK) and myeloma cells, in particular Chinese Hamster Ovary (CHO) cells.

Cell Culture In some embodiments, the cells used in practising the invention are capable of growing in suspension cultures. As used herein, suspension-competent cells are those that can grow in suspension without making large, firm aggregates, i.e., cells that are monodisperse or grow in loose aggregates with only a few cells per aggregate.

The cells used in practicing the invention may be adhesion cells (also known as anchorage-dependent or attachment-dependent cells). As used herein, adhesion cells are those that need to adhere or anchor themselves to a suitable surface for propagation and growth.

### Cell Viability

Cell viability is a determination of living or dead cells, based on a total cell sample. Cell death can be divided into two different events, necrosis and apoptosis. Necrosis is the death of cells as a result of disease or injury. The cells swell, their plasma membranes become disrupted, and the cell contents are released into the extracellular space, where they often trigger an inflammatory response. The necrosis process is unregulated. Apoptosis on the other hand is a mechanism that allows cells to self-destruct when stimulated by the appropriate trigger. It may be initiated when a cell is no longer needed, when a cell becomes a threat to the organism's health, or for other reasons.

Testing for cell viability usually involves looking at a sample cell population and staining the cells or applying chemicals to show which are living and which are dead. There are numerous tests and methods for measuring cell viability.

In most normal and viable eukaryotic cells the negatively charged phospholipid phosphatidylserine (PS) is located on the cytosolic side of the plasma membrane lipid bilayer. The phosphatidylserine is redistributed from the inner leaflet to the outer leaflet during apoptosis of eukaryotic cells. Annexin V is a Ca++ dependent phospholipid-binding protein that react with phosphatidylserine (PS). Apoptosis can be detected in flow cytometry by incubating cells with fluorescently labelled Annexin V. In early phases of necrosis the cell membrane becomes disrupted and Annexin V can access the PS in the inner leaflet of these cells as well.

A method for detecting membrane permeability is the common dye exclusion method. Fluorescent, DNA-binding probes as propidium iodide (PI) and 7-amino actinomycin D (7-AAD) enter dying cells and stain the DNA. A dye exclusion method that does not require flow cytometer knowledge is the dye exclusion procedure for microscopy using trypan blue and a hemacytometer.

Other ways of determining viability is based on the ATP contents of the cells, which is an indicator of metabolic active cells. The CellTiter-GLO kit transforms ATP to luminescence, which is proportional to the viability of the cells. This method is relative and it is not possible to study individual cells.

Large-scale animal cell cultures are used extensively in the production of therapeutic proteins by the pharmaceutical industry and by biotechnology companies. Cells that experience medium depletion will die via apoptosis (starvation-induced apoptosis), and only at high stress levels (e.g. sudden drop in pH or high concentrations of toxins) cells die via necrosis.

FVIII and FVIIIa does by its nature bind to activated platelets by their exposure of phosphatidylserines, and it is on this cell surface the FVIIIa/FIXa complex activates FX in vivo. Phophatidylserines on apoptotic cells or membrane fragments from necrotic cells are also bound by FVIII. Production of FVIII in an animal cell culture will lead to binding of FVIII to dying cells and the FVIII protein are consequently “trapped” there.

### Cell Medium

The term “cell culture medium” (or simply “medium”) refers to a nutrient solution used for growing mammalian cells that typically provides at least one component from one or more of the following categories: (1) salts of e.g. sodium, potassium, magnesium, and calcium contributing to the osmolality of the medium; (2) an energy source, usually in the form of a carbohydrate such as glucose; (3) all essential amino acids, and usually the basic set of twenty amino acids; (4) vitamins and/or other organic compounds required at low concentrations; and (5) trace elements, where trace elements are defined as inorganic compounds that are typically required at very low concentrations, usually in the micromolar range. The nutrient solution may optionally be supplemented with one or more of the components from any of the following categories: (a) hormones and other growth factors such as, for example, insulin, transferrin, and epidermal growth factor; and (b) hydrolysates of protein and tissues. Preferably, the cell culture medium does not contain any components of animal origin.

In one embodiment, the medium lacks animal-derived components and lacks proteins (“protein-free”). Media lacking animal-derived components and/or proteins are available from commercial suppliers, such as, for example, Sigma, JRH Biosciences, Gibco, Hyclone and Gemini.

In one embodiment, the cell culture medium is serum free. Preferably, the cell culture medium comprises less than 0.25% serum by volume. In a further embodiment, the medium is totally free from proteins (“protein-free”) as well as lacking animal- derived components.

Preferably, in the methods of the invention, a mammalian cell capable of expressing a human Factor VIII polypeptide is cultured in a cell medium free from animal-derived components and is contacted with an agent that binds to phosphatidylserine, such as lactadherin, by adding said agent to the medium. Preferably, in the methods of the invention, a mammalian cell capable of expressing a human Factor VIII polypeptide is cultured in a cell medium free from animal-derived components and is contacted with an agent that binds to phosphatidylserine, such as annexin V, by adding said agent to the medium. In connection with the present invention, said agent can be added to the culture medium at a concentration of between 0.01 and 100 μM, such as e.g. 0.01-50 μM, 0.01-25 μM, 0.01-10 μM, or 0.01-1 μM, 0.01-0.1 μM, 0.1-100 μM, 0.1-50 μM, 0.1-25 μM, 0.1-10 μM, 0.1-1 μM, 1-100 μM, 1-50 μM, 1-25 μM, 1-10 μM, 10-100 μM, 10-50 μM, or 10-25 μM.

In the methods of the present invention, one or more agents that bind to phosphatidylserine may be contacted with the culture cells by adding into the culture medium. The cell medium may also comprise additional agents that reduce binding of Factor VIII to the cell membrane and/or improve the stability or titer of Factor VIII. For example, agents such as Ortho-Phospho-L-serine (OPLS), anti-apoptotic proteins or heparin may be added to the culture medium.

In one embodiment of the present invention a cell culture medium is provided that is serum free and comprises i) a compound selected from lactadherin, annexin V, an antiphospholipid antibody and Factor VIII light chain, and ii) Ortho-Phospho-L-serine (OPLS) or an anti-apoptotic protein, for use in the methods of the invention. Most preferably, the culture medium is free from animal-derived components and comprises lactadherin and OPLS. The culture medium free from animal-derived components of the invention may comprise Factor VIII light chain and OPLS. Typically, the concentration of OPLS in the culture medium is between 1 M and 100 mM, between 10 M and 50 mM, between 100 M and 50 mM, between 1 mM and 50 mM or between 1 mM and 30 mM.

### Large-Scale Culture Conditions

The invention is particularly relevant for large-scale production. By the term “large-scale production” is meant production involving a culture vessel of at least 100 L. In preferred embodiments, however, the scale is typically at least 250 L, such as at least 500 L, e.g. at least 1000 L or even 5000 L or more. The term “large-scale” may be used interchangeably with the terms “industrial-scale” and “production-scale”.

Contact of Cell Culture with an Agent that Binds to Phosphatidylserine

In one embodiment of the present invention, one or more agents that bind phosphatidylserine are contacted with the culture cells producing Factor VIII. Further, one or more additional agents that reduce binding of Factor VIII to the cell membrane and/or improve the stability or titer of Factor VIII may be contacted with the culture cells in addition to the agent that binds phosphatidylserine.

Any agent capable of binding to phosphatidylserine may be used in the method of the present invention. The agent that binds to phosphatidylserine may be or may comprise a polypeptide, antibody, antibody fragment, polynucleotide, small molecule or other agent.

Typically, the agent that binds to phosphatidylserine is capable of reducing the binding of Factor VIII to phosphatidylserine on the cell membrane. The agent may compete with Factor VIII to bind to phosphatidylserine. Preferred agents are those that reduce the binding of Factor VIII to the cell membrane by at least 10%, at least 20%, at least 30%, at least 40%, at least 50%, at least 60%, at least 70%, at least 80%, at least 90% compared to the binding seen in the absence of the agent.

The agent that binds phosphatidylserine preferably increases the yield of Factor VIII isolated from the cell culture. Typically, the yield of Factor VIII is isolated from the cell culture medium. Therefore, preferred agents are those that increase the yield of Factor VIII, or the amount of Factor VIII released into the culture medium by at least 10%, at least 20%, at least 30%, at least 40%, at least 50%, at least 60%, at least 70%, at least 80%, at least 90% compared to the yield or the release of Factor VIII in the absence of the agent.

A competitive binding assay may be used to identify agents that competitively bind to phosphatidylserine on the cell membrane. This technique involves use of unlabelled and labelled anlaytes which compete for phosphatidylserine on the cell membrane. The general technique of competitive binding assays is well known in the art. The assay gives signals which decrease as the concentration of the target analyte increases. A competitive assay approach may be used to detect an agent that binds to phosphatidylserine by its ability to compete with Factor VIII for binding to the cell membrane. For example, an agent that binds to phosphatidylserine and is suitable for use in the methods of the invention may be identified by its ability in a competition assay, to reduce the binding of Factor VIII to the cell membrane by at least 50%.

The annexin V (annexin A5 or vascular anticoagulant alpha protein) for use in the methods of the invention may be a naturally occurring annexin V polypeptide or a fragment or variant thereof that is still capable of binding to phosphatidylserine. The variant polypeptide may be a species homologue, such as a mammalian homologue (typically human, primate or mouse, rat or other rodent homologue). Preferably, annexin V is human annexin V. A suitable human annexin V polypeptide may comprise, consist of or consist essentially of the amino acid sequence of SEQ ID NO:2. A suitable annexin V sequence may be a fragment or variant of this sequence that is capable of binding to phosphatidylserine. For example, a variant of annexin V may be a substitution, deletion or addition variant or a fragment thereof.

Preferably, the fragment or variant of a naturally occurring annexin V is capable of competing with Factor VIII for binding sites on the cell membrane. Typically, the fragment or variant retains at least one cell membrane binding domain. The fragment or variant may also retain at least one protein binding domain required for formation of a protein-protein complex that blocks binding of Factor VIII to the cell membrane.

Lactadherin for use in the methods of the invention may be a naturally occurring lactadherin polypeptide or a fragment or variant thereof that is still capable of binding to phosphatidylserine. The variant polypeptide may be a species homologue, such as a mammalian homologue (typically human, primate or mouse, rat or other rodent homologue). Preferably, the lactadherin is human lactadherin. A suitable human lactadherin polypeptide may comprise, consist of or consist essentially of the amino acid sequence of SEQ ID NO:3. A suitable lactadherin sequence may be a fragment or variant of this sequence that is capable of binding to phosphatidylserine. For example, a variant of lactadherin may be a substitution, deletion or addition variant or a fragment thereof.

A factor VIII light chain for use in the methods of the invention may comprise domains A3-C1-C2 of Factor VIII. A factor VIII light chain may be produced by recombinantly expressing nucleic acid encoding Factor VIII domains A3-C1-C2. Alternatively, or additionally, a Factor VIII light chain may be produced by proteolytic processing of at the B-A3 junction of a Factor VIII polypeptide.

A fragment or variant of Factor VIII light chain may also be used in the methods of the invention provided the fragment or variant is still capable of binding to phosphatidylserine. Typically, the fragment or variant is capable of competing with Factor VIII for binding sites on the cell membrane. Typically, the fragment or variant retains at least one cell membrane binding domain. For example, the fragment or variant may comprise domain C2. Most preferably, the fragment or variant comprises domains C1 and C2. In particular, the fragment or variant comprises the C2 domain sequence represented by SEQ ID NO: 6 (amino acids 2173 to 2332 of human Factor VIII), or a variant of that C2 domain comprising up to 20, up to 10, up to 5, or up to 2 amino acid substitutions and/or deletions. The fragment or variant may comprise the amino acid sequence2303 to 2332 of human Factor VIII C2 domain or a variant of that sequence comprising 1, 2, 3, 4, 5, 6 or 7 amino acid substitutions and/or deletions.

An antiphospholipid antibody suitable for use in the methods of the invention includes any antibody that binds to one or more phospholipids including phosphatidylserine. The antiphospholipid antibody may bind to phosphatidylserine and one or more other phospholipids including but not limited to an amphipathic phospholipid, a lipid bilayer phospholipid, a phosphoglyceride, a phosphatidate, a phosphatidyl choline, a phosphatidyl ethanolamine, a phosphatidyl inositol, a diphosphatidyl glycerol or a sphingomyelin. Typically, the antiphospholipid antibody is able to compete for, reduce, or inhibit the binding of Factor VIII to the cell membrane.

The antibody may be a human, mouse, rat, goat, rabbit, guinea pig, chicken, sheep or horse antibody. Preferably, the antiphospholipid antibody is a human, humanized, chimeric, rat or mouse antibody.

A suitable antiphospholipid antibody sequence may be a fragment or variant of this sequence that is capable of binding to phosphatidylserine. For example, a variant of a naturally occurring antiphospholipid antibody may be a substitution, deletion or addition variant or a fragment thereof.

Polypeptides and variants and fragments thereof, as discussed above may be provided by expression from a nucleic acid molecule. The invention thus also relates to polynucleotides comprising nucleic acid sequences which encode annexin V, lactadherin, Factor VIII light chain, or an anti-phospholipid antibody or any derivative, fragment or variant thereof.

The agent may be provided in the culture medium at a concentration sufficient to reduce or inhibit binding of Factor VIII to the cell membrane. Typically, the agent is capable of increasing the concentration of Factor VIII in the culture medium surrounding the culture cells. Preferably, the agent that binds phosphatidylserine is contacted with the culture cells by adding to the cell culture medium at a concentration of between 0.001 and 1000 μM, between 0.01 and 500 μM, between 0.01 and 100 μM, between 0.01 and 10 μM or between 0.1 and 100 μM.

The agent that binds phosphatidylserine is added to the cell culture medium during or after a period of culturing the cells that express Factor VIII but before isolation of Factor VIII from the culture medium. Typically, the cells that express Factor VIII are cultured for at least 6 hours, at least 12 hours, at least 24 hours, at least 48 hours, at least 4 days or at least 10 days before isolation of Factor VIII from the culture medium. The agent that binds phosphatidylserine may be contacted with the culture cells simultaneously, at substantially the same time, or at a different time to when the cells are initially contacted with the culture medium. The agent may be added repeatedly to the culture medium, for example after regular intervals, or each time fresh medium is contacted with the culture cells. The agent may be added immediately prior to isolating Factor VIII from the culture medium.

One or more, two or more, three or more, four or more agents that bind to phosphatidylserine may be contacted with the culture cells expressing Factor VIII. For example, two agents selected from annexin V, lactadherin, factor VIII light chain and an antiphospholipid antibody may be used in the methods of the invention.

The amount of Factor VIII polypeptide in the culture medium may be measured by techniques well known in the art. The Factor VIII polypeptide may be labelled, for example using a radioisotope, radionucleotide, fluorescent moiety such as GFP, enzyme, affinity tag such as biotin, histidine or GST, epitope tag, antibody, or polynucleotide. If Factor VIII is labelled, then yield may be calculated by isolating and detecting the labelled Factor VIII in the culture medium, for example by spectroscopic, photochemical, radiochemical, biochemical, immunochemical, chemical or electrochemical means that are known in the art.

If unlabelled, Factor VIII can be isolated from the culture medium as described below, using techniques well known in the art. Purification of Factor VIII polypeptides may involve affinity chromatography on an anti-Factor VIII antibody column and activation by proteolytic cleavage.

In a first aspect, the present invention thus relates to a method for the production of a Factor VIII polypeptide, which method comprises:
a) culturing a mammalian cell capable of expressing a Factor VIII polypeptide under conditions such that the said polypeptide is expressed; and
b) during or after step (a), contacting the said cell with an agent that binds to phosphatidylserine.

In one embodiment, said method further comprises the step of harvesting the Factor VIII polypeptide at a point in time where the viability of the cells is at least 80%, preferably at least 85%, most preferably at least 90%, and most preferably at least 95%.

In another embodiment, said method further comprises the step of harvesting the Factor VIII polypeptide after 2-3 days, or after 2-4 days, such as e.g. after 2 days, or after 3 days or after 4 days.

In another embodiment, the mammalian cell is cultured in a cell culture medium wherein the Factor VIII polypeptide is a human Factor VIII polypeptide.

In another embodiment, the agent is contacted with the mammalian cell by i) co-expressing the agent with Factor VIII, or ii) adding the agent to a culture medium in which the cell is cultured. The cell may be a transiently or a stably transformed cell.

In another embodiment, the agent is a protein that specifically binds to phosphatidylserine, preferably lactadherin, annexin V, an antiphospholipid antibody or a Factor VIII light chain.

In another embodiment, the lactadherin, annexin V or Factor VIII light chain is added or co-expressed at a concentration of 0.01 to 100 μM.

In another embodiment, one, two, three or more agents capable of binding to phosphatidylserine on the cell membrane are contacted with the mammalian cell.

In another embodiment, lactadherin, annexin V, antiphospholipid antibody or Factor VIII light chain is contacted with the mammalian cell together with Ortho-Phospho-L-serine (OPLS) or an anti-apoptotic protein.

In another embodiment, the mammalian cell is cultured in a cell culture medium free from animal-derived components. Alternatively the method according to the invention further comprises isolating the Factor VIII polypeptide and optionally formulating the Factor VIII polypeptide into a pharmaceutical composition.

In another embodiment, the Factor VIII polypeptide is isolated from a cell culture medium in which the mammalian cell is cultured, substantially without reduction of the viability of the cells, wherein preferably at least 75%, or 80%, or 85%, or 90% of the cells remain viable.

In another embodiment, after isolating the Factor VIII polypeptide, the same cell is used in a method according to any one of the preceding claims.

Another aspect of the present invention relates to a cell culture medium that is serum free and comprises i) an agent selected from lactadherin, annexin V, an antiphospholipid antibody and Factor VIII light chain, and ii) Ortho-Phospho-L-serine (OPLS) or an anti-apoptotic protein.

Another aspect of the present invention relates to use of an agent capable of binding to phosphatidylserine for increasing the yield of Factor VIII that can be isolated from a mammalian cell culture.

### EXAMPLES

Binding Assays

### Method

The affinity of purified B-domain deleted factor VIII (BDD-FVIII) (kindly provided by J. Karlsson and L. Thim, Novo Nordisk A/S) to the cell membrane of HEK293 cells was investigated by a homologous competition assay using ¹²⁵I-BDD-FVIII and unlabelled BDD-FVIII. Cells were washed once in PBS+1% BSA. 5×10⁵ cells were distributed to a microtiter well and the plate was cooled to 4° C. During blocking of the cell surface, binding of BDD-FVIII was examined. A constant concentration of ¹²⁵I-FVIII (0.5 nM) was added simultaneously with either Annexin V (0.5 μM, Sigma), Ortho-Phospho-L-serine (20 mM, Sigma), Heparin (100 μg mL-1, Leo Pharmaceuticals), and Receptor Associated Protein (RAP) 0.5 μM (kindly provided by H. H. Petersen, Novo Nordisk A/S) at 4° C. to prevent endocytosis.

The plate was incubated at 4° C. for 2 hours with gentle shaking. After centrifugation unbound (non-membrane attached) ¹²⁵I-FVIII was removed and cells were washed twice in ice-cold assay buffer (10 mM HEPES, 150 mM NaCl, 4 mM KCl, 11 mM glucose, 5 mM CaCl₂, 1 mg ml⁻¹ BSA, pH 7.4). Surface bound ¹²⁵I-FVIII was counted on a gamma-counter. The experiments were performed twice in triplicate. Non-specific binding was estimated in the presence of 12000× excess of unlabeled BDD Factor VIII.

In an attempt to determine potent inhibitors of the cell membrane interaction, four proteins known for their specific effect in either: 1) blocking phosphatidylserine (annexin V), 2) interacting with the C2 domain of FVIII (OPLS), 3) interacting with receptors that facilitate internalisation followed by degradation such as LRP (Lipoprotein receptor-related protein) and HSPGs (heparin sulphate proteoglycans) were tested (RAP, heparin).

### Results

The results are shown in Table 2 below.

TABLE 2

| | % of total binding | stdev |
|---|---|---|
| 0.5 nM | 95.9 | 2.5 |
| 125I-FVIII | | |
| unspecific binding | 11.9 | 0.7 |
| Annexin V 500 nM | 28.5 | 3.1 |
| OPLS 20 mM | 68.4 | 5.2 |
| Heparin 100 ug/mL | 86.8 | 7.7 |
| RAP 500 nM | 106.6 | 6.9 |

Annexin V reduced membrane attached FVIII by ˜70% and Ortho-Phospho-L-serine (OPLS) reduced the membrane attached FVIII by ˜30%. Heparin showed a small but not significant effect. RAP showed no effect.

Because annexin V was able to reduce the membrane binding most efficiently we continued investigating other compounds that would also inhibit PS binding of FVIII on the cell surface. This is described in the next experiment.

### FVIII Membrane Displacement Cell Cultures

### Method

CHO DUKX B11 cells stably expressing BDD-FVIII were set up in a high density (8×10⁶ cells mL⁻¹) in a 50 mL filter tubes (TPP, Switzerland) in serum free medium. The additives mentioned below (lactadherin, Factor VIII light chain and/or OPLS) were added to the culture medium and the cells were incubated for 24 hours following assaying of the culture fluid and the membrane bound fraction.

### Results

The results are shown in Tables 3A and 3B below.

TABLE 3A

| | supernatant | | wash | |
|---|---|---|---|---|
| | average | stdev | average | stdev |
| Control | 3829 | 464 | 2342 | 559 |
| 20 mM OPLS | 6347 | 2070 | 1691 | 542 |
| F8 LC 1.38 μM | 9733 | 660 | 1919 | 881 |
| F8 LC 1.38 μM/20 mM | 13625 | 1025 | 382 | 45 |
| OPLS | | | | |

TABLE 3B

| | supernatant | | wash | |
|---|---|---|---|---|
| | average | stdev | average | stdev |
| Control | 3829 | 464 | 2342 | 559 |
| 20 mM OPLS | 6347 | 2070 | 1691 | 542 |
| Lactadherin 0.15 μM | 8085 | 1546 | 1406 | 215 |
| Lactadherin 0.15 μM/20 mM | 8489 | 1439 | 1138 | 193 |
| OPLS | | | | |

By adding OPLS the activity of FVIII increases from 4000 to 6000 mU/mL in the culture medium and the activity on the membrane does not drop proportionally. This could illustrate the stabilising effect of the OPLS added. The addition of lactadherin further increases the amount of FVIII in the culture medium and also a decline in membrane bound FVIII is observed. When both compounds (lactadherin and OPLS) are added only a small increase is seen compared to the addition of lactadherin alone. Compared to the control culture the amount of FVIII in the fluidic phase is increased 2.2 fold.

A similar tendency is observed when the FVIII LC is added to the medium. However, the increase of the FVIII yield in the fluidic phase is considerably higher compared to the addition of lactadherin. This is possible due to the much higher concentrations of FVIII LC added, which causes a more complete competition of FVIII from the cell membranes. In this case the addition of FVIII LC and OPLS contributes even further to the fluidic phase FVIII fraction and the overall improvement is above 3-fold.

### Co-Expression Experiments

Cell Culture

HEK293 cells were maintained in commercial FreeStyle medium supplemented with 50 U/mL penicillin and 50 ug/mL streptomycin. Cells were grown as suspension cells in shakers and incubated at 37° C. under 5% CO2 and 95% relative humidity conditions.

Cells were seeded at a density of 3×105 cells/mL and passaged every 3-4 days. For transfection experiments the cell culture was scaled up until the target density was reached. Viable and total cell concentrations were evaluated by Cedex (Innovartis) analysis. The instrument uses image analysis software for automated cell counting and viable cells were identified based on their ability to exclude trypan blue.

Transient Transfection

Plasmid DNA was transfected into HKB11 cells by 293 fectin following the manufacturer's recommendations. Conditioned medium was harvested on indicated days following gentle centrifugation of the suspension culture. The cell pellet was resuspended in FreeStyle medium containing 0.5M NaCl and after gentle centrifugation, a sample, representing the FVIII attached to the cell membrane was taken. Samples were stored at −80° C. until analysis.

Factor VIII Activity and Antigen Analysis

FVIII coagulation activity was measured by a two-stage chromogenic assay (Coamatic Factor VIII analysis kit, Chromogenix). Factor VIII:Ag assay was performed using polyclonal antibodies from Affinity Biologicals (F8C-EIA). Both assays were done following the manufactures instructions and with in-house B-domain deleted affinity purified Factor VIII as standard.

TABLE 4A
An expression plasmid encoding F8 was transiently expressed in HKB11 cells with co-
expression of the indicated plasmids. Only F8 was expressed in the transfection with
pcDNA3.1. coa values are given in mU/ml. Experiments were performed in duplicate.

| | | Harvest day | | |
|---|---|---|---|---|
| Co-expression | | 3 | 4 | 5 |
| construct | Sample type | coa (mU/ml) | | |
| Lactadherin | wash | 640 +/− 116 | 3575 +/− 558 | 3510 +/− 46 |
| | supernatant | 5448 +/− 122 | 1044 +/− 425 | 327 +/− 13 |
| hGH- | wash | 1220 +/− 482 | 3424 +/− 239 | 4325 +/− 930 |
| Lactadherin | supernatant | 3611 +/− 216 | 844 +/− 502 | 298 +/− 42 |
| hGH- | wash | 1456 +/− 27 | 3120 +/− 6 | 4117 +/− 281 |
| Lactadherin-C1C2 | supernatant | 2929 +/− 1054 | 1340 +/− 203 | 556 +/− 84 |
| hFc- | wash | 2116 +/− 173 | 3368 +/− 159 | 3868 +/− 284 |
| Lactadherin-C1C2 | supernatant | 1566 +/− 95 | 429 +/− 52 | 595 +/− 91 |
| pcDNA3.1 | wash | 3082 +/− 409 | 5023 +/− 414 | 6319 +/− 600 |
| (empty vector) | supernatant | 621 +/− 69 | 413 +/− 57 | 506 +/− 16 |

TABLE 4B
Same experiment as Table 4A. FVIII ELISA values are
given in ng/ml. Experiments were performed in duplicate.

| | | Harvest day | | |
|---|---|---|---|---|
| Co-expression | | 3 | 4 | 5 |
| construct | Sample type | FVIII ELISA (ng/ml) | | |
| Lactadherin | wash | 67 +/− 0 | 278 +/− 37 | 287 +/− 33 |
| | supernatant | 530 +/− 113 | 270 +/− 54 | 108 +/− 119 |
| hGH- | wash | 150 +/− 82 | 277 +/− 14 | 327 +/− 45 |
| Lactadherin | supernatant | 605 +/− 243 | 262 +/− 97 | 132 +/− 52 |
| hGH- | wash | 156 +/− 13 | 268 +/− 6 | 294 +/− 18 |
| Lactadherin-C1C2 | supernatant | 540 +/− 42 | 221 +/− 13 | 122 +/− 4 |
| hFc- | wash | 182 +/− 6 | 258 +/− 3 | 308 +/− 9 |
| Lactadherin-C1C2 | supernatant | 302 +/− 11 | 148 +/− 27 | 112 +/− 0 |
| pcDNA3.1 | wash | 268 +/− 34 | 449 +/− 26 | 446 +/− 38 |
| (empty vector) | supernatant | 116 +/− 129 | 129 +/− 132 | 120 +/− 120 |

TABLE 4C
Same experiment as Table 4A. Cell counts are
given in 106 c/ml and viability in % living cells of
total cells. Experiments were performed in duplicate.

| Co- | Cell count | | | |
|---|---|---|---|---|
| expression | and | Harvest day | | |
| construct | viability | 3 | 4 | 5 |
| Lactad- | Cell count | 1.54 +/− 0.082 | 1.17 − 0.062 | 1.29 +/− 0.186 |
| herin | viability | 91 +/− 0.1 | 81 +/− 0.2 | 66 +/− 4.9 |
| hGH- | Cell count | 1.56 +/− 0.016 | 1.31 +/− 0.202 | 1.49 +/− 0.076 |
| Lactad- | viability | 92 +/− 1.7 | 83 +/− 1.3 | 68 +/− 3.4 |
| herin | | | | |
| hGH- | Cell count | 1.92 +/− 0.14 | 1.38 +/− 0.235 | 1.53 +/− 0.030 |
| Lactad | viability | 91 | 82 | 62 |
| herin- | | | | |
| C1C2 | | | | |
| hFc- | Cell count | 1.66 +/− 0.010 | 1.54 +/− 0.006 | 1.33 +/− 0.098 |
| Lactad- | viability | 91 +/− 0.2 | 84 +/− 0.8 | 65 +/− 2.4 |
| herin- | | | | |
| C1C2 | | | | |
| pcDNA3.1 | Cell count | 1.85 +/− 0.023 | 1.4107 − 0.053 | 1.53 +/− 0.047 |
| (empty | viability | 94 +/− 0.8 | 85 +/− 1.3 | 67 +/− 0.3 |
| vector) | | | | |

Results

An expression plasmid encoding F8 was transiently co-expressed with lactadherin in HKB11 cells, as well as with lactadherin fused to the C-terminal of human growth hormone (hGH-Lactadherin). F8 was also co-expressed with hGH-LactadherinC1C2 (the C1C2 domains of Lactadherin fused to the C-terminal of hGH) and hFc-LactadherinC1C2 (the C1C2 domains of Lactadherin fused to the C-terminal of human Fc). The fusion partners were selected for their ability to possibly facilitate increased expression of lactadherin or its domains. It was found that on HKB11 cells, F8 was effectively displacement on day 3, by Lactadherin and hGH-Lactadherin and also to a lesser extent by hGH-LactadherinC1C2 and hFc-LactadherinC1C2 (Table 4A and 4B). However, on day 4 when viability of the cells had dropped to ˜80%, from ˜90% on day 3, Lactadherin co-expression could no longer keep F8 in the supernatant, and F8 was primarily located on the cell surface equivalent to expression with F8 alone. (Table 4A, 4B and 4C)

Conclusion

These results show that, on day 3, co-expression with Lactadherin (or Lactadherin fused to an N-terminal fusion partner, or the C1C2 domains of Lactadherin together with an N-terminal fusion partner) can change F8 from being localized on the cell membrane, to being located in the supernatant. We believe this is a result of Lactadherin blocking PS binding sites on the HKB11 cells, and thereby prevent F8 to bind to the same sites. As a consequence F8 is effectively displaced into the supernatant. The results also show that on day 4, co-expression with Lactadherin has no longer any effect on F8 localization. We believe this is a consequence of the relatively low viability, ˜80%, which probably correlate with the number of PS binding sites increasing the number of sites that the expressed amounts of Lactadhering are able to shield. And therefore, on day 4, there are likely free PS binding sites on the cells that F8 will bind.

TABLE 5
Clones stably transfected with lactadherin (SEQ ID No 7)
and FVIII encoding plasmids. “COA” is a measure of FVIII
activity (this type of chromogenic assay (e.g. COATEST
SPFVIII assay #82408663 from Chromogenix) is
well known in the art). A high level of COA in the supernatant
thus means that a high proportion of FVIII is present in the
supernatant. The levels of COA measured in the “wash” equals
the amount of FVIII extracted when the cells are washed
with high salt medium for releasing FVIII bound or attached to cell
membranes. Low “wash” COA levels thus indicate
that not much FVIII is attached to cell membranes. Eight
clones have been selected for further characterization (table 6).

| First screen | COA (mU/ml), | COA (mU/ml), | COA (mU/ml), |
|---|---|---|---|
| Clone name | supernatant | wash | total |
| CS282_F4 | 12405 | 1099 | 13504 |
| CS282_F184 | 11733 | 1022 | 12755 |
| CS282_F120 | 11420 | 1070 | 12490 |
| CS282_F223 | 11498 | 716 | 12214 |
| CS282_F258 | 11458 | 716 | 12174 |
| CS282_F167 | 10638 | 982 | 11619 |
| CS282_F130 | 10715 | 895 | 11610 |
| CS282_F143 | 10365 | 1050 | 11415 |
| CS282_F194 | 10405 | 854 | 11259 |
| CS282_F66 | 10135 | 781 | 10916 |
| CS282_F43 | 10018 | 895 | 10912 |
| CS282_F263 | 10755 | 0 | 10755 |
| CS282_F133 | 10600 | 0 | 10600 |
| CS282_F257 | 9518 | 994 | 10511 |
| CS282_F118 | 9633 | 668 | 10301 |
| CS282_F205 | 9555 | 743 | 10298 |
| CS282_F53 | 9403 | 696 | 10099 |
| CS282_F138 | 9940 | 0 | 9940 |
| CS282_F100 | 8678 | 1248 | 9926 |
| CS282_F209 | 9288 | 608 | 9896 |
| CS282_F137 | 9213 | 668 | 9881 |
| CS282_F245 | 9135 | 689 | 9824 |
| CS282_F92 | 9020 | 641 | 9661 |
| CS282_F12 | 8755 | 809 | 9564 |
| CS282_F24 | 8793 | 743 | 9536 |
| CS282_F33 | 8718 | 724 | 9442 |
| CS282_F321 | 8375 | 1011 | 9386 |
| CS282_F18 | 8565 | 798 | 9363 |
| CS282_F127 | 8640 | 641 | 9281 |
| CS282_F333 | 8263 | 909 | 9172 |
| CS282_F325 | 8300 | 826 | 9126 |
| CS282_F278 | 8000 | 781 | 8781 |
| CS282_F29 | 8000 | 641 | 8641 |
| CS282_F303 | 7775 | 752 | 8527 |
| CS282_F132 | 8490 | 0 | 8490 |
| CS282_F59 | 8338 | 0 | 8338 |
| CS282_F240 | 7663 | 608 | 8271 |
| CS282_F271 | 8263 | 0 | 8263 |
| CS282_F259 | 7475 | 662 | 8137 |
| CS282_F281 | 7400 | 641 | 8041 |
| CS282_F288 | 7925 | 0 | 7925 |
| CS282_F81 | 7775 | 0 | 7775 |
| CS282_F36 | 6808 | 724 | 7532 |
| CS282_F113 | 7475 | 0 | 7475 |
| CS282_F72 | 7215 | 0 | 7215 |
| CS282_F39 | 7178 | 0 | 7178 |
| CS282_F78 | 7105 | 0 | 7105 |
| CS282_F276 | 6845 | 0 | 6845 |
| CS282_F247 | 6185 | 608 | 6793 |
| CS282_F254 | 6405 | 0 | 6405 |

TABLE 6
Eight clones were selected from table 5. Clone “1C9” is a control, wherein cells
have been stably transfected with FVIII only and not lactadherin. The amount of FVIII present
in the supernatant is significantly increased in clones stably transfected with lactadherin
plasmids. According to the last two columns, there is not as much active FVIII (using COA
activity assays) present in the supernatant as FVIII antigen (measured using standard FVIII
ELISA). This ratio can be improved by addition of stabilizers such as OPLS.

| | COA | COA | COA | ELISA | ELISA | ELISA | Lactdherin | ratio | |
|---|---|---|---|---|---|---|---|---|---|
| | (Sup) | (Wash) | total | (Sup) | (Wash) | total | ELISA | COA (sup)/ | |
| Clones | mU/ml | mU/ml | mU/ml | ng/ml | ng/ml | ng/ml | OD450 | COA (wash) | [symbol] |
| F4 | 32164 | 15684 | 47847 | 10015 | 595 | 10610 | 2.11 | 2.05 | [symbol] |
| F59 | 23453 | 15197 | 38650 | 3794 | 761 | 4556 | 1.57 | 1.54 | [symbol] |
| F120 | 22963 | 12635 | 35598 | 4033 | 814 | 4848 | 1.88 | 1.82 | [symbol] |
| F133 | 22963 | 12635 | 35598 | 4033 | 814 | 4848 | 1.63 | 1.82 | [symbol] |
| F167 | 27137 | 15271 | 42408 | 6509 | 709 | 7218 | 2.31 | 1.78 | [symbol] |
| F184 | 23042 | 14670 | 37712 | 4916 | 780 | 5696 | 2.04 | 1.57 | [symbol] |
| F223 | 23619 | 15109 | 38728 | 6175 | 718 | 6893 | 1.89 | 1.56 | [symbol] |
| F263 | 24480 | 15346 | 39826 | 6778 | 630 | 1877 | 1.92 | 1.60 | [symbol] |
| 1C9 | 5195 | 10196 | 15391 | 649 | 771 | 1420 | 0.02 | 0.51 | [symbol] |

[symbol]

indicates data missing or illegible when filed

Stable Cell Line Generation

The cell line 1C9, that stably express BDD-FVIII was transfected with plasmid #2140. #2140 encodes a fusion construct consisting of the FLAG epitope followed by lactadherin, and also carries the neomycin resistance gene. The 1C9 cells were electroporated and selected with 500 ug/ml G418. Transfection and selection was carried out in the serum-free medium B-CM208.

Screening

After three weeks of selection, the cells were cloned by limited dilution and transferred to 24 well plates. Supernatant and “wash” samples were collected from fifty clones in these 24 well plates. As seen from Table (fifty clones) all clones appeared to express more FVIII in the supernatant than in the wash fraction. The wash fraction was prepared by subjecting the cells to a solution of B-CM208 with addition of 0.55 M NaCl.

In table (ten clones) some of these clones, and the 1C9 clone, have been grown in 30 ml medium in shaker flasks over a period of a couple of weeks. Shown is the average values of COA activity and ELISA yield. The results from an ELISA assay against the FLAG-epitope are also shown. It can be seen that the supernatant from the 1C9 clone, as excepted, do not show any response in the FLAG ELISA. It can also be seen, that the other clones, that have been stably selected with G418, express varying levels of the FLAG epitope. Thus, we see correlation between expression of FLAG-lactadherin and an increased localization of FVIII in the supernatant.

Methods:

Flag ELISA

Supernatant or wash samples were applied to ANTI-FLAG High Sensitivity M2 coated 96-well plates (Cat. P2973-1EA, SIGMA). After incubation for 60 min, the plates were washed in PBS, and an antibody against lactadherin (Cat. H00004240-D01P, ABNOVA) was added. After another incubation for 60 min, plates were washed and developed with an anti-rabbit-HRP conjugated antibody, and absorbance was read at 450 nm.

1. A method for the production of a Factor VIII polypeptide, said method comprising:
a) culturing a mammalian cell capable of expressing a Factor VIII polypeptide under conditions such that the said polypeptide is expressed; and
b) during or after step (a), contacting the said cell with an agent that binds to phosphatidylserine.

2. The method according to claim 1, wherein said method further comprises the step of harvesting the Factor VIII polypeptide at a point in time where the viability of the cells is at least 80%.

3. The method according claim 1, wherein said method further comprises the step of harvesting the Factor VIII polypeptide after 2-3 days.

4. The method according to claim 1 wherein the mammalian cell is cultured in a cell culture medium and wherein the Factor VIII polypeptide is a human Factor VIII polypeptide.

5. The method according to claim 1, wherein the agent is contacted with the mammalian cell by i) co-expressing the agent with Factor VIII, or ii) adding the agent to a culture medium in which the cell is cultured.

6. The method according to claim 1, wherein the agent is a protein that specifically binds to phosphatidylserine, selected from the group consisting of lactadherin, annexin V, an antiphospholipid antibody and a Factor VIII light chain.

7. The method according to claim 6, wherein the lactadherin, annexin V or Factor VIII light chain is added or co-expressed at a concentration of 0.01 to 100 μM.

8. The method according to claim 1, wherein one, two, three or more agents capable of binding to phosphatidylserine on the cell membrane are contacted with the mammalian cell.

9. The method according to claim 6, wherein lactadherin, annexin V, antiphospholipid antibody or Factor VIII light chain is contacted with the mammalian cell together with Ortho-Phospho-L-serine (OPLS) or an anti-apoptotic protein.

10. The method according to claim 1, wherein the mammalian cell is cultured in a cell culture medium free from animal-derived components, wherein the method further comprises isolating the Factor VIII polypeptide and formulating the Factor VIII polypeptide into a pharmaceutical composition.

11. The method according to claim 10, wherein the Factor VIII polypeptide is isolated from a cell culture medium in which the mammalian cell is cultured, substantially without reduction of the viability of the cells, wherein at least 85% of the cells remain viable.

12. The method according to claim 10, wherein after isolating the Factor VIII polypeptide, the same mammalian cell is used in a method for the production of a Factor VIII polypeptide, comprising a) culturing said mammalian cell under conditions such that Factor VIII polypeptide is expressed; and b) during or after step (a), contacting said cell with an agent that binds to phosphatidylserine.

13. A cell culture medium that is serum free and comprises i) an agent selected from lactadherin, annexin V, an antiphospholipid antibody and Factor VIII light chain, and ii) Ortho-Phospho-L-serine (OPLS) or an anti-apoptotic protein.

14. (canceled)

15. A method for increasing the yield of Factor VIII that is isolated from a mammalian cell culture, comprising the use of an agent that binds to phosphatidylserine.
